(19) United States
(12) Patent Application Publication
(10) Pub. No.: US 2025/0250220 A1
(43) Pub. Date: Aug. 7, 2025

(54) CATALYTIC METHODS FOR CARBONYLATION OF ESTERS

(71) Applicants: THE UNIVERSITY OF NORTH CAROLINA AT CHAPEL HILL, Chapel Hill, NC (US); EASTMAN CHEMICAL COMPANY, Kingsport, TN (US)

(72) Inventors: Alexander James Minden Miller, Chapel Hill, NC (US); Javier Martinez Grajeda, Kingsport, TN (US); Changho Yoo, Chapel Hill, NC (US); Nathan Mitchell West, Kingsport, TN (US); Xin Yi See, Kingsport, TN (US); Steven Thomas Perri, Kingsport, TN (US); Dawn Chamaine Mason, Raleigh, NC (US); Chris David Meade, Blountville, TN (US); Drew Cunningham, Carrboro, NC (US)

(21) Appl. No.: 18/695,162

(22) PCT Filed: Sep. 26, 2022
(86) PCT No.: PCT/US2022/044766

§ 371 Date: Mar. 25, 2024

Related U.S. Application Data
(60) Provisional application No. 63/248,727, filed on Sep. 27, 2021.

(51) Int. Cl. C07C 51/56; B01J 31/22

(52) U.S. Cl. CPC C07C 51/56; B01J 31/2273; B01J 2231/34; B01J 2531/822; B01J 2531/827; B01J 2531/847; C07C 2531/22

(57) ABSTRACT

Disclosed are improved catalytic carbonylation methods. In general, the methods are suitable for carbonylating a variety of esters in the presence of carbon monoxide or a source thereof and a catalyst system comprising a transition metal-carbene complex; or a neutral carbene or salt thereof together with a transition metal compound; and a halide source for use as a halide promoter.

### CROSS-REFERENCE TO RELATED APPLICATIONS

This application claims priority to U.S. Provisional Application No. 63/248,727, filed Sep. 27, 2021, the entirety of which is incorporated into this application by reference.

### BACKGROUND

Catalytic carbonylation refers to a catalytic reaction in which carbon monoxide is added to an organic substrate. Carbonylation is widely used in industry to produce a variety of commercially useful products such as anhydrides, carboxylic acids, and esters. To achieve desirable yields using inexpensive metal catalysts, however, the required catalyst loading is typically high. Commonly used transition-metal catalysts can be very expensive and undergo sudden and steep price surges based on availability and demand.

Common catalysts for carbonylation of esters are phosphine-based transition metal catalysts, such as catalysts including one or more triphenylphosphine ligands. Triphenylphosphine transition-metal catalysts routinely achieve sub-optimal turnover numbers such that the amount of ligand or metal used is relatively high. This makes such catalysts unattractive alternatives to more commonly used, yet highly expensive, transition metal complexes. Accordingly, there is a need in the art for improved catalytic methods for carbonylating substrates such as esters. These needs and others are met by the present disclosure.

### SUMMARY

In one aspect, disclosed is a method comprising carbonylating an ester in a reactor comprising carbon monoxide or a source thereof in the presence of a catalyst system; wherein the ester has a structure represented by Formula (I):

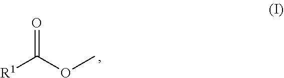

wherein R¹ is hydrocarbyl; wherein the catalyst system comprises: a) at least one of: i) a transition metal-carbene complex; or ii) a carbene ligand, or a salt thereof, and a transition metal compound; and b) a halide source.

In another aspect, disclosed is a reaction medium comprising: a) an ester having a structure represented by Formula (I):

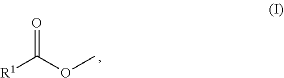

wherein R¹ is hydrocarbyl; b) carbon monoxide or a source thereof; and c) a catalyst system comprising: i) at least one of: 1) a transition metal-carbene complex; or 2) a carbene ligand, or a salt thereof, and a transition metal compound; and ii) a halide source.

In a further aspect, disclosed is a method comprising carbonylating an ester in a reactor comprising carbon monoxide or a source thereof in the presence of a catalyst system; wherein the ester has a structure represented by Formula (I):

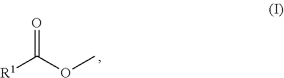

wherein R¹ is hydrocarbyl; wherein the catalyst system comprises: a) at least one of:
i) a transition metal-N-heterocyclic carbene complex having a structure represented by Formula (IX-M):

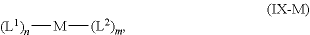

wherein n is an integer ranging from 1-2 and m is an integer ranging from 1-5, provided that when n is 2, m is an integer ranging from 1-4; L¹ is a ligand having a structure represented by the formula:

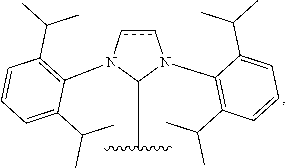

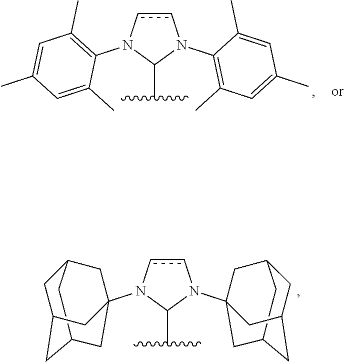

wherein the dashed line (---) represents an optional covalent bond; wherein M is a Group 8, 9, or 10 transition metal, such as nickel, rhodium, or iridium, for example; and
wherein each instance of L² is independently —CO or halide;
ii) an N-heterocyclic carbene ligand having a structure represented by Formula (IX), (X), or (XI):

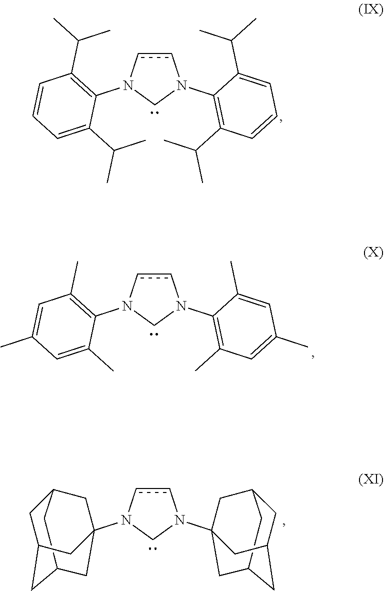

wherein the dashed line (---) represents an optional covalent bond; or
iii) a salt of an N-heterocyclic carbene ligand having a structure represented by Formula (IX-S), (X-S), or (XI-S):

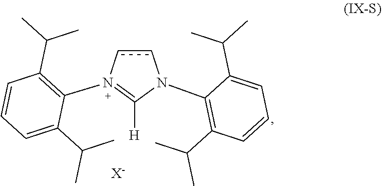

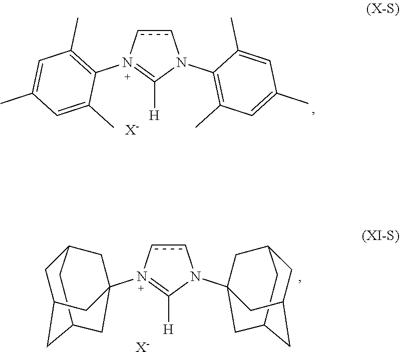

wherein the dashed line (---) represents an optional covalent bond; and wherein X is halide, BF₄, or PF₆; wherein the N-heterocyclic carbene ligand, when present, or the salt of the carbene ligand, when present, is present together with a transition metal compound comprising a Group 8, 9, or 10 transition metal, such as nickel, rhodium, or iridium; and b) a halide source.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Disclosed are components that can be used to perform the disclosed methods. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and products. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

While aspects of this disclosure can be described and claimed in a particular statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### A. Definitions

Listed below are definitions of various terms. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where the event or circumstance occurs and instances where it does not.

“Carbonylation” means a reaction in which carbon monoxide is introduced into an organic substrate, either using carbon monoxide gas or a source thereof. For example, methyl propionate can be carbonylated in the presence of carbon monoxide or a source thereof to produce acetic propionic anhydride along with other reaction products including acetic acid and methyl acetate.

“Hydrocarbyl” encompasses C1-C24 alkyl, C2-C24 alkenyl, and C2-C24 alkynyl, whether linear or branched. A hydrocarbyl can be optionally substituted, in which at least one hydrogen of the hydrocarbyl has been replaced with a group that is not hydrogen, such as halide groups, hydroxyl groups, ether groups, thiol groups, thiol ether groups, carboxylic acid groups, carboxylic acid ester groups, phosphoric acid groups, phosphoric acid ester groups, sulfonic acid groups, sulfonic acid ester groups, nitro groups, cyano groups, cycloalkyl groups, cycloalkenyl groups, cycloalkynyl groups, aryl groups, heteroaryl groups, among others.

“Alkyl” means a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. “Alkyl” can be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

“Cycloalkyl” means a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. “Heterocycloalkyl” is a non-aromatic carbon-based ring type of cycloalkyl group, where at least one of the carbon atoms of the ring is replaced with a heteroatom such as, but not limited to, nitrogen, oxygen, sulfur, or phosphorus. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol.

“Bicyclic cycloalkyl” or “bicyclic heterocycloalkyl” refers to a compound in which two or more cycloalkyl or heterocycloalkyl groups are fused together. Non-limiting examples of bicyclic cycloalkyl groups include without limitation (1r,4r)-bicyclo[2.1.1]hexane, (1s,4s)-bicyclo[2.2.1]heptane, (1R,6S)-bicyclo[4.2.0]octane, adamantane, and the like. Non-limiting examples of bicyclic heterocycloalkyl groups include without limitation any of the foregoing groups in which at least one of the carbon atoms is replaced with a heteroatom such as nitrogen, oxygen, sulfur, or phosphorus.

“Alkenyl” means a hydrocarbon having from 2 to 24 carbons with a structural formula containing at least one carbon-carbon double bond. Asymmetric structures such as (A¹A²)C═C(A³A⁴) are intended to include both the E and Z isomers. The alkenyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Cycloalkenyl” means a non-aromatic carbon-based ring composed of at least three carbon atoms and containing at least one carbon-carbon double bound, i.e., C═C. Examples of cycloalkenyl groups include cyclopropenyl, cyclobutenyl, cyclopentenyl, cyclopentadienyl, cyclohexenyl, cyclohexadienyl, norbornenyl, among others. The term “heterocycloalkenyl” is a type of cycloalkenyl group and is included within the meaning of the term “cycloalkenyl,” where at least one of the carbon atoms of the ring is replaced with a heteroatom such as nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Alkynyl” means a hydrocarbon group of 2 to 24 carbon atoms with a structural formula containing at least one carbon-carbon triple bond. The alkynyl group can be unsubstituted or substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Cycloalkynyl” means a non-aromatic carbon-based ring composed of at least seven carbon atoms and containing at least one carbon-carbon triple bound. Examples of cycloalkynyl groups include cycloheptynyl, cyclooctynyl, cyclononynyl, and the like. The term “heterocycloalkynyl” is a type of cycloalkenyl group and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, among others.

“Aryl” means a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. In addition, the aryl group can be a single ring structure or comprise multiple ring structures that are either fused ring structures or attached via one or more bridging groups such as a carbon-carbon bond. For example, aryl can include biaryl in which two aryl groups are bound together via a fused ring structure, as in naphthalene, or are attached via one or more carbon-carbon bonds, as in biphenyl.

“Heteroaryl” means an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further non-limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

“Halide” means F, Cl, Br, or I.

“Transition metal” can refer to the IUPAC definition, which defines a transition metal as an element whose atom has a partially filed d sub-shell, or which can give rise to cations with an incomplete d sub-shell. Alternatively, “transition metal” can refer to any element in the d-block of the periodic table, which includes Group 3-12 metals. In one aspect, “transition metal” can be a Group 8, 9, or 10 element, such as nickel, rhodium, or iridium.

“Carbene” means a molecule containing a neutral carbon atom with a valence of two and two unshared valence electron, i.e., a “—C:”-containing molecule. A “carbene salt” or “salt of a carbene” refers to a compound in which the carbene has been converted to salt with a positively charged atom and a negatively charged counterion.

“Syngas” means a gaseous mixture comprising carbon monoxide, hydrogen, and in some instances carbon dioxide.

“Reactor” means any suitable vessel useful for performing the catalytic reaction methods. The reactor can be a smaller, lab-scale reactor, or a larger commercial scale reactor. Smaller reactors include, without limitation, steel pressure reactors containing glass or TEFLON (PTFE) liners. In other aspects, the reactor can be a Hastelloy autoclave having a suitable volume. In some aspects, the reactor can be equipped with an infrared spectroscopy probe for in situ monitoring of the reaction mixture.

“Molar ratio” means the moles of one substance relative to the moles of another substance.

“Turnover number” or “TON” means the moles of a reaction product divided by the moles of a precatalyst or catalyst added to the reactor.

“Partial pressure” means the pressure of a constituent gas in the atmosphere of the reaction medium, which is the notional pressure of that constituent gas if that gas occupied the entire volume of the original mixture at the same temperature. Partial pressures of a gas in a reactor can be measured according to methods known in the art.

### B. Catalytic Carbonylation

Previous research on transition metal catalysts such as nickel catalysts for carbonylation reactions have typically been reported only in conjunction with tertiary phosphine or amine ligands. Such ligands are prone to methylation or oxidation, leading to catalyst decomposition or side reactions. High loadings of transition metal are required, which defeats the purpose of moving to a low-cost metal. The present methods feature catalysts or precatalyst systems comprising carbene ligands, such as N-heterocyclic carbene (NHC) ligands. Such catalyst systems exhibit higher activity at lower catalyst loadings, showing promise for the development of a process that could compete with industry-standard catalysts. The homogeneous nature of these catalysts lends itself well for rapid implementation into the existing infrastructure for large-scale carbonylation processes.

The selectivity of the catalysts for carbonylation (vs. hydrogenation) also optionally allows for the use of syngas with hydrogen gas present along with carbon monoxide. The large number of available carbene (e.g., NHC) ligands provides a convenient method to tune reactivity. One advantage of the disclosed catalytic methods is that free carbene (e.g., NHC) ligands or air-stable salts (protonated carbenes, e.g., protonated NHCs) can be used in conjunction with simple transition metal salts or compounds. Alternatively, an isolated transition metal-carbene complex can be used. The described methods are useful for carbonylation reactions used in the synthesis of various carboxylic acids, anhydrides, esters, alkyl acetates, and other large-scale commodity chemicals.

In general, the carbonylation reaction converts esters such as alkyl esters to anhydrides (with other products such as acetic acid and acetyl esters) under an atmosphere of carbon monoxide. In one aspect, the method comprises carbonylating an ester in a reactor comprising carbon monoxide or a source thereof in the presence of a catalyst system. The catalyst system comprises at least one of: a transition metal-carbene complex; or a carbene ligand, or a salt thereof, and a transition metal compound. Thus, the method allows for the use of isolated carbene complexes as well as catalytic precursors (neutral carbene ligands and salts of carbene ligands) that allow for the formation of the carbene catalyst in situ. In addition, the catalyst system comprises a halide source such as an alkyl halide.

When free carbene ligands or salts thereof are used, the ligand or salt can be present in the catalyst system in an amount equal to or in excess of the transition metal compound. In some aspects, for example, the ligand or salt thereof can be present in the catalyst system in a 1:1-10:1 molar ratio relative to the transition metal compound. For example, the ligand or salt thereof can be present in the catalyst system in a 2:1, 5:1, or 10:1 molar ratio relative to the transition metal compound, or in other words, two, five, or ten equivalents of ligand relative to the transition metal precursor.

The catalytic reactions can be carried out at a variety of suitable temperatures. In one aspect, carbonylation is carried out at a temperature of at least 50° C. In a further aspect, carbonylation is carried out at a temperature of at least 180° C., e.g., 180° C.-200° C. In a further aspect, carbonylation is carried out at a temperature of at least 200° C., e.g., 200° C.-220° C.

Carbonylation can generally be carried out at a suitable time which can depend on a variety of factors. Reaction products, however, can be monitored to determine when the reaction mixture should be quenched if necessary. Suitable reaction times include for example 3-24 hours, e.g., 10-15 hours, or much longer times when carried out on large industrial scales. In general, the reaction can be carried out for any suitable time as indicated by methods for measuring reaction progress and completion. In addition, the carbonylation reaction can be implemented as part of a batch or continuous process.

### 1. Ester Starting Materials and Reaction Products

A variety of esters can be carbonylated using the disclosed methods. In one aspect, the ester has a structure represented by Formula (I):

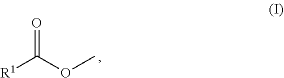

wherein R¹ is hydrocarbyl.

The hydrocarbyl group at R¹ can be C1-C24 alkyl, C2-C24 alkenyl, and C2-C24 alkynyl, whether linear or branched, as defined above. The hydrocarbyl group can be optionally substituted as defined above, in which at least one hydrogen of the hydrocarbyl has been replaced with a group that is not hydrogen, such as halide groups, hydroxyl groups, ether groups, thiol groups, thiol ether groups, carboxylic acid groups, carboxylic acid ester groups, phosphoric acid groups, phosphoric acid ester groups, sulfonic acid groups, sulfonic acid ester groups, nitro groups, cyano groups, cycloalkyl groups, cycloalkenyl groups, cycloalkynyl groups, aryl groups, heteroaryl groups, among others.

In one aspect, the hydrocarbyl group at R¹ can be an alkyl ester, i.e., a branched or unbranched saturated hydrocarbon comprising 1-24 carbon atoms, not including any carbon atoms present on optional substituents. Thus, the alkyl ester can be substituted or unsubstituted. For example, in some aspects, the alkyl ester can be substituted with one or more groups including but not limited to alkyl, cycloalkyl, alkoxy, amino, ether, halide, hydroxy, nitro, silyl, sulfo-oxo, or thiol. The alkyl ester can be entirely acyclic or comprise one or more cyclic groups.

In one aspect, the hydrocarbyl at R¹ can be C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl. When R¹ comprises more than two carbon atoms, R¹ can be branched or unbranched, for example branched or unbranched C3-C4 alkyl, branched or unbranched C3-C5 alkyl, branched or unbranched C3-C6 alkyl, branched or unbranched C3-C7 alkyl, branched or unbranched C3-C8 alkyl, branched or unbranched C3-C9 alkyl, branched or unbranched C3-C10 alkyl, and the like up to and including branched or unbranched C3-C24 alkyl. In certain specific aspects, R¹ is C1-C20 alkyl, C1-C10 alkyl, or C2-C3 alkyl.

Non-limiting examples of hydrocarbyl groups at R¹ include methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. In one specific aspect, R¹ is methyl, ethyl, or isopropyl. Thus, specific alkyl ester starting materials include without limitation methyl acetate, methyl propionate, methyl butyrate, and methyl isobutyrate.

The ester can be carbonylated in neat form or with the addition of a suitable solvent, such as an organic solvent. Solvents can be readily determined by one skilled in the art.

Esters of Formula (I), such as alkyl esters, can be converted to a variety of reaction products, typically including a predominance of an anhydride corresponding to Formula (I-P):

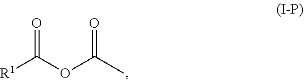

where R¹ is defined above with reference to the ester starting material of Formula (I). In addition, other carboxylic acids and acetates can also form. For example, when carbonylating methyl propionate, major reaction products include acetic propionic anhydride, in addition to the symmetric anhydride (propionic anhydride), acetic acid, and methyl acetate. As one of skill in the art will appreciate, the nature of the reaction product mixture depends generally on the starting ester of Formula (I).

### 2. Carbon Monoxide and Sources Thereof

In one aspect, the reactor comprises carbon monoxide or a source thereof. In a further aspect, the carbon monoxide is present in a syngas composition comprising hydrogen gas. In addition, any suitable source of carbon monoxide gas can be used, including precursor materials that can form carbon monoxide in the reactor, for example under increased pressure. Examples of precursor materials that can form carbon monoxide in situ include carbon dioxide, metal carbonyls, formic acid derivatives, and methanol, among others. These sources of carbon monoxide can be desirable for minimizing any toxicity and transportation problems resulting from gaseous carbon monoxide.

In general, the carbon monoxide in the reactor will be pressurized. For example, in one aspect, the carbon monoxide is present in the reactor at a partial pressure of at least 20 bar. In a further aspect, the carbon monoxide is present in the reactor at a partial pressure of 20-50 bar. In some aspects, the carbon monoxide or source thereof, or reactor, is substantially free of water, or in some aspects, free of water.

### 3. Catalyst Systems

The catalyst system generally comprises at least one of: (i) a transition metal-carbene complex; or (ii) a carbene ligand, or a salt thereof, and a transition metal compound; as well as a halide promoter, which can be any halide source, such as an alkyl halide. The transition metal compound, when present in the catalyst system, can be any suitable transition metal compound such as a compound comprising a Group 8, 9, or 10 transition metal. Non-limiting examples include NiI₂, NiCl₂, Ni(OAc)₂, IrI₃, IrCl₃, Ir(OAc) 3, RhI₃, RhCl₃, and Rh(OAc)₃.

a. Catalyst and Catalytic Precursors

In general, the catalyst systems comprise a transition metal-carbene complex as a catalyst or a catalyst precursor mixture that includes for example a carbene ligand, or a salt of the carbene ligand, together with a transition metal compound. When present in a catalyst system, the catalyst precursor mixture will form a catalyst from the carbene ligand or salt thereof and the transition metal compound. In other words, the transition metal-carbene complex can be generated in situ in the reaction medium.

In one aspect, the transition metal-carbene complex, when present, is a transition metal-N-heterocyclic carbene complex. Similarly, the carbene ligand, when present, can be an N-heterocyclic carbene. It should be understood that in general, when the transition metal-carbene complex is preset, additional free carbene or carbene salt can be added to the reactor. The salt of the carbene ligand, when present, can be an N-heterocyclic carbene salt. In one aspect, the transition metal-N-heterocyclic carbene complex, when present, or the transition metal compound, when present, comprises a Group 8, 9, or 10 transition metal. For example, transition metals that can be present in the carbene complexes or precursor transition metal compounds include nickel, rhodium, or iridium. In one aspect, the transition metal present in the transition metal compound or carbene complex is nickel.

One advantage of the disclosed catalytic methods is that far less catalyst is required to achieve commercially viable turnover numbers (TONs) and reaction yields. Thus, in one aspect, prior to carbonylation, the ester and the transition metal-carbene complex or the transition metal compound are present in the reactor at a molar ratio ranging from 100:1 to 10,000:1 (ester:transition metal-carbene complex or transition metal compound). In a further aspect, prior to carbonylation, the ester and the transition metal-carbene complex or the transition metal compound are present in the reactor at a molar ratio ranging from 250:1 to 10,000:1 (ester:transition metal-carbene complex or transition metal compound). In a further aspect, prior to carbonylation, the ester and the transition metal-carbene complex or the transition metal compound are present in the reactor at a molar ratio ranging from 500:1 to 10,000:1 (ester:transition metal-carbene complex or transition metal compound). In a further aspect, prior to carbonylation, the ester and the transition metal-carbene complex or the transition metal compound are present in the reactor at a molar ratio ranging from 750:1 to 10,000:1 (ester:transition metal-carbene complex or transition metal compound). In a further aspect, prior to carbonylation, the ester and the transition metal-carbene complex or the transition metal compound are present in the reactor at a molar ratio ranging from 1,000:1 to 10,000:1 (ester:transition metal-carbene complex or transition metal compound). In a further aspect, prior to carbonylation, the carbene ligand or salt thereof and the transition metal-carbene complex or the transition metal compound are present in the reactor at a molar ratio ranging from 1:1 to 10:1 (carbene ligand or salt thereof:transition metal-carbene complex or transition metal compound).

In one aspect, the carbene ligand, when present, has a structure represented by Formula (II):

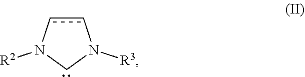

wherein the dashed line (---) represents an optional covalent bond; and wherein R² and R³ are independently selected from aryl, heteroaryl, cycloalkyl, heterocycloalkyl, bicyclic cycloalkyl, or bicyclic heterocycloalkyl.

In a further aspect, the salt of the carbene ligand, when present, has a structure represented by Formula (II-S):

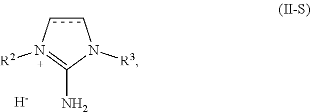

wherein the dashed line (---) represents an optional covalent bond; and wherein R² and R³ are independently selected from aryl, heteroaryl, cycloalkyl, heterocycloalkyl, bicyclic cycloalkyl, or bicyclic heterocycloalkyl; and wherein X is halide, BF₄, or PF₆.

In a further aspect, the transition metal-carbene complex, when present, has a structure represented by Formula (II-M):

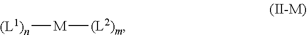

wherein n is an integer ranging from 1-2 and m is an integer ranging from 1-5, provided that when n is 2, m is an integer ranging from 1-4; L¹ is a ligand having a structure represented by the formula:

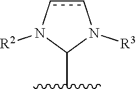

wherein the dashed line (---) represents an optional covalent bond; wherein R² and R³ are independently selected from aryl, heteroaryl, cycloalkyl, heterocycloalkyl, bicyclic cycloalkyl, or bicyclic heterocycloalkyl; wherein M is a Group 8, 9, or 10 transition metal; and wherein each instance of L² is independently —CO or halide.

In one aspect, the carbene ligand, when present, has a structure represented by Formula (III):

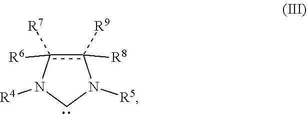

wherein each instance of the dashed line (---) represents an optional covalent bond; wherein R⁴ and R⁵ are independently selected from aryl, heteroaryl, cycloalkyl, heterocycloalkyl, bicyclic cycloalkyl, or bicyclic heterocycloalkyl; wherein R⁶ and R⁸ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R⁶ and R⁸ can together form an aryl, heteroaryl, cycloalkyl, heterocycloalkyl, bicyclic cycloalkyl, or bicyclic heterocycloalkyl ring; and wherein R⁷ and R⁹, when present, are independently selected from hydrogen, halide, C1-C4 alkyl, aryl, heteroaryl, cycloalkyl, heterocycloalkyl, bicyclic cycloalkyl, or bicyclic heterocycloalkyl.

In a further aspect, the salt of the carbene ligand, when present, has a structure represented by Formula (III-S):

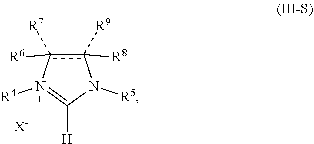

wherein each instance of the dashed line (---) represents an optional covalent bond; wherein R⁴ and R⁵ are independently selected from aryl, heteroaryl, cycloalkyl, heterocycloalkyl, bicyclic cycloalkyl, or bicyclic heterocycloalkyl; wherein R⁶ and R⁸ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R⁶ and R⁸ can together form an aryl, heteroaryl, cycloalkyl, heterocycloalkyl, bicyclic cycloalkyl, or bicyclic heterocycloalkyl ring; wherein R⁷ and R⁹, when present, are independently selected from hydrogen, halide, C1-C4 alkyl, aryl, heteroaryl, cycloalkyl, heterocycloalkyl, bicyclic cycloalkyl, or bicyclic heterocycloalkyl; and wherein X is halide, BF₄, or PF₆.

In a further aspect, the transition metal-carbene complex, when present, has a structure represented by Formula (III-M):

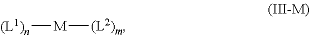

wherein n is an integer ranging from 1-2 and m is an integer ranging from 1-5, provided that when n is 2, m is an integer ranging from 1-4; L¹ is a ligand having a structure represented by the formula:

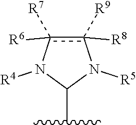

wherein each instance of the dashed line (---) represents an optional covalent bond; wherein R⁴ and R⁵ are independently selected from aryl, heteroaryl, cycloalkyl, heterocycloalkyl, bicyclic cycloalkyl, or bicyclic heterocycloalkyl; wherein R⁶ and R⁸ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R⁶ and R⁸ can together form an aryl, heteroaryl, cycloalkyl, heterocycloalkyl, bicyclic cycloalkyl, or bicyclic heterocycloalkyl ring; wherein R⁷ and R⁹, when present, are independently selected from hydrogen, halide, C1-C4 alkyl, aryl, heteroaryl, cycloalkyl, heterocycloalkyl, bicyclic cycloalkyl, or bicyclic heterocycloalkyl; wherein M is a Group 8, 9, or 10 transition metal; and wherein each instance of L² is independently —CO or halide.

In one aspect, the carbene ligand, when present, has a structure represented by Formula (IV):

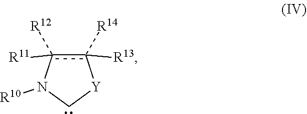

wherein each instance of the dashed line (---) represents an optional covalent bond; wherein R¹⁰ is selected from aryl, heteroaryl, cycloalkyl, heterocycloalkyl, bicyclic cycloalkyl, or bicyclic heterocycloalkyl; wherein R¹¹ and R¹³ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R¹¹ and R¹³ can together form an aryl, heteroaryl, cycloalkyl, heterocycloalkyl, bicyclic cycloalkyl, or bicyclic heterocycloalkyl ring; wherein R¹² and R¹⁴, when present, are independently selected from hydrogen, halide, C1-C4 alkyl, aryl, heteroaryl, cycloalkyl, heterocycloalkyl, bicyclic cycloalkyl, or bicyclic heterocycloalkyl; and wherein Y is selected from oxygen and sulfur.

In a further aspect, the salt of the carbene ligand, when present, has a structure represented by Formula (IV-S):

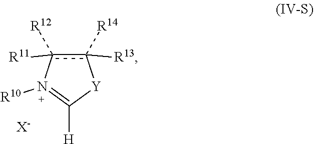

wherein each instance of the dashed line (---) represents an optional covalent bond; wherein R¹⁰ is selected from aryl, heteroaryl, cycloalkyl, heterocycloalkyl, bicyclic cycloalkyl, or bicyclic heterocycloalkyl; wherein R¹¹ and R¹³ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R¹¹ and R¹³ can together form an aryl, heteroaryl, cycloalkyl, heterocycloalkyl, bicyclic cycloalkyl, or bicyclic heterocycloalkyl ring; wherein R¹² and R¹⁴, when present, are independently selected from hydrogen, halide, C1-C4 alkyl, aryl, heteroaryl, cycloalkyl, heterocycloalkyl, bicyclic cycloalkyl, or bicyclic heterocycloalkyl; wherein Y is selected from oxygen and sulfur; and wherein X is halide, BF₄, or PF₆.

In a further aspect, wherein the transition metal-carbene complex, when present, has a structure represented by Formula (IV-M):

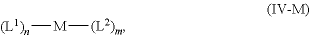

wherein n is an integer ranging from 1-2 and m is an integer ranging from 1-5, provided that when n is 2, m is an integer ranging from 1-4; L¹ is a ligand having a structure represented by the formula:

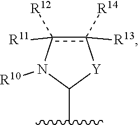

wherein each instance of the dashed line (---) represents an optional covalent bond; wherein R¹⁰ is selected from aryl, heteroaryl, cycloalkyl, heterocycloalkyl, bicyclic cycloalkyl, or bicyclic heterocycloalkyl; wherein R¹¹ and R¹³ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R¹¹ and R¹³ can together form an aryl, heteroaryl, cycloalkyl, heterocycloalkyl, bicyclic cycloalkyl, or bicyclic heterocycloalkyl ring; wherein R¹² and R¹⁴, when present, are independently selected from hydrogen, halide, C1-C4 alkyl, aryl, heteroaryl, cycloalkyl, heterocycloalkyl, bicyclic cycloalkyl, or bicyclic heterocycloalkyl; wherein Y is selected from oxygen and sulfur; wherein M is a Group 8, 9, or 10 transition metal; and wherein each instance of L² is independently —CO or halide.

In one aspect, the carbene ligand, when present, has a structure represented by Formula (V-A) or (V-B):

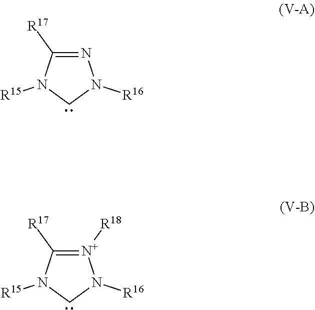

wherein R¹⁵ and R¹⁶ are independently selected from aryl, heteroaryl, cycloalkyl, heterocycloalkyl, bicyclic cycloalkyl, or bicyclic heterocycloalkyl; wherein R¹⁷ is selected from hydrogen, halide, C1-C4 alkyl, aryl, heteroaryl, cycloalkyl, heterocycloalkyl, bicyclic cycloalkyl, or bicyclic heterocycloalkyl; and wherein R¹⁸, when present, is selected from hydrogen, halide, C1-C4 alkyl, aryl, heteroaryl, cycloalkyl, heterocycloalkyl, bicyclic cycloalkyl, or bicyclic heterocycloalkyl.

In a further aspect, the salt of the carbene ligand, when present, has a structure represented by Formula (V-S-A) or (V-S-B):

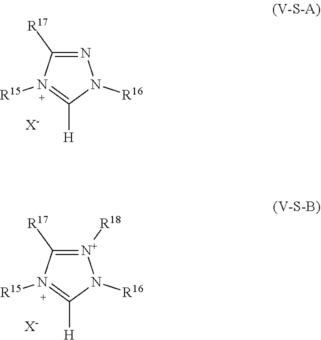

wherein R¹⁵ and R¹⁶ are independently selected from aryl, heteroaryl, cycloalkyl, heterocycloalkyl, bicyclic cycloalkyl, or bicyclic heterocycloalkyl; wherein R¹⁷ is selected from hydrogen, halide, C1-C4 alkyl, aryl, heteroaryl, cycloalkyl, heterocycloalkyl, bicyclic cycloalkyl, or bicyclic heterocycloalkyl; wherein R¹⁸, when present, is selected from hydrogen, halide, C1-C4 alkyl, aryl, heteroaryl, cycloalkyl, heterocycloalkyl, bicyclic cycloalkyl, or bicyclic heterocycloalkyl; and wherein X is halide, BF₄, or PF₆.

In a further aspect, wherein the transition metal-carbene complex, when present, has a structure represented by Formula (V-M):

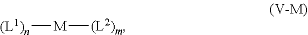

wherein n is an integer ranging from 1-2 and m is an integer ranging from 1-5, provided that when n is 2, m is an integer ranging from 1-4; L¹ is a ligand having a structure represented by the Formula (V-M-A) or (V-M-B):

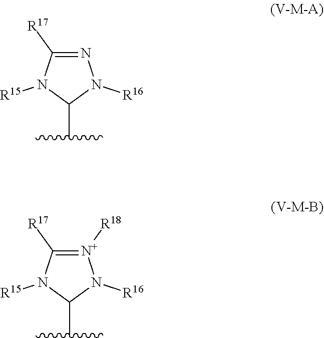

wherein R¹⁵ and R¹⁶ are independently selected from aryl, heteroaryl, cycloalkyl, heterocycloalkyl, bicyclic cycloalkyl, or bicyclic heterocycloalkyl; wherein R¹⁷ is selected from hydrogen, halide, C1-C4 alkyl, aryl, heteroaryl, cycloalkyl, heterocycloalkyl, bicyclic cycloalkyl, or bicyclic heterocycloalkyl; wherein R¹⁸, when present, is selected from hydrogen, halide, C1-C4 alkyl, aryl, heteroaryl, cycloalkyl, heterocycloalkyl, bicyclic cycloalkyl, or bicyclic heterocycloalkyl; wherein M is a Group 8, 9, or 10 transition metal; and wherein each instance of L² is independently —CO or halide.

In one aspect, the carbene ligand, when present, has a structure represented by Formula (VI):

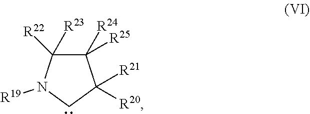

wherein R¹⁹ is selected from aryl, heteroaryl, cycloalkyl, heterocycloalkyl, bicyclic cycloalkyl, or bicyclic heterocycloalkyl; wherein R²⁰ and R²¹ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁰ and R²¹ can together form a cycloalkyl, heterocycloalkyl, bicyclic cycloalkyl, or bicyclic heterocycloalkyl ring; wherein R²² and R²³ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²² and R²³ can together form a cycloalkyl, heterocycloalkyl, bicyclic cycloalkyl, or bicyclic heterocycloalkyl ring; wherein R²⁴ and R²⁵ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁴ and R²⁵ can together form a cycloalkyl, heterocycloalkyl, bicyclic cycloalkyl, or bicyclic heterocycloalkyl ring.

In a further aspect, the salt of the carbene ligand, when present, has a structure represented by Formula (VI-S):

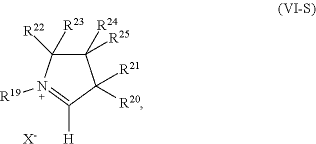

wherein R¹⁹ is selected from aryl, heteroaryl, cycloalkyl, heterocycloalkyl, bicyclic cycloalkyl, or bicyclic heterocycloalkyl; wherein R²⁰ and R²¹ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁰ and R²¹ can together form a cycloalkyl, heterocycloalkyl, bicyclic cycloalkyl, or bicyclic heterocycloalkyl ring; wherein R²² and R²³ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²² and R²³ can together form a cycloalkyl, heterocycloalkyl, bicyclic cycloalkyl, or bicyclic heterocycloalkyl ring; wherein R²⁴ and R²⁵ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁴ and R²⁵ can together form a cycloalkyl, heterocycloalkyl, bicyclic cycloalkyl, or bicyclic heterocycloalkyl ring; and wherein X is halide, BF₄, or PF₆.

In a further aspect, the transition metal-carbene complex, when present, has a structure represented by Formula (VI-M):

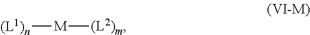

wherein n is an integer ranging from 1-2 and m is an integer ranging from 1-5, provided that when n is 2, m is an integer ranging from 1-4; L¹ is a ligand having a structure represented by the formula:

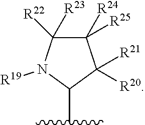

wherein R¹⁹ is selected from aryl, heteroaryl, cycloalkyl, heterocycloalkyl, bicyclic cycloalkyl, or bicyclic heterocycloalkyl; wherein R²⁰ and R²¹ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁰ and R²¹ can together form a cycloalkyl, heterocycloalkyl, bicyclic cycloalkyl, or bicyclic heterocycloalkyl ring; wherein R²² and R²³ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²² and R²³ can together form a cycloalkyl, heterocycloalkyl, bicyclic cycloalkyl, or bicyclic heterocycloalkyl ring; wherein R²⁴ and R²⁵ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁴ and R²⁵ can together form a cycloalkyl, heterocycloalkyl, bicyclic cycloalkyl, or bicyclic heterocycloalkyl ring; wherein M is a Group 8, 9, or 10 transition metal; and wherein each instance of L² is independently —CO or halide.

In one aspect, the carbene ligand, when present, has a structure represented by Formula (VII):

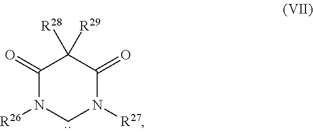

wherein R²⁶ and R²⁷ are independently selected from aryl, heteroaryl, cycloalkyl, heterocycloalkyl, bicyclic cycloalkyl, or bicyclic heterocycloalkyl; wherein R²⁸ and R²⁹ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁸ and R²⁹ can together form a cycloalkyl, heterocycloalkyl, bicyclic cycloalkyl, or bicyclic heterocycloalkyl ring.

In a further aspect, the salt of the carbene ligand, when present, has a structure represented by Formula (VII-S):

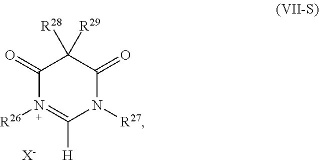

wherein R²⁶ and R²⁷ are independently selected from aryl, heteroaryl, cycloalkyl, heterocycloalkyl, bicyclic cycloalkyl, or bicyclic heterocycloalkyl; wherein R²⁸ and R²⁹ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁸ and R²⁹ can together form a cycloalkyl, heterocycloalkyl, bicyclic cycloalkyl, or bicyclic heterocycloalkyl ring; and wherein X is halide, BF₄, or PF₆.

In a further aspect, the transition metal-carbene complex, when present, has a structure represented by Formula (VII-M):

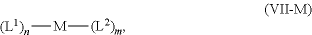

wherein n is an integer ranging from 1-2 and m is an integer ranging from 1-5, provided that when n is 2, m is an integer ranging from 1-4; L¹ is a ligand having a structure represented by the formula:

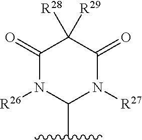

wherein R²⁶ and R²⁷ are independently selected from aryl, heteroaryl, cycloalkyl, heterocycloalkyl, bicyclic cycloalkyl, or bicyclic heterocycloalkyl; wherein R²⁸ and R²⁹ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R²⁸ and R²⁹ can together form a cycloalkyl, heterocycloalkyl, bicyclic cycloalkyl, or bicyclic heterocycloalkyl ring; wherein M is a Group 8, 9, or 10 transition metal; and wherein each instance of L² is independently —CO or halide.

In one aspect, the carbene ligand, when present, has a structure represented by Formula (VIII):

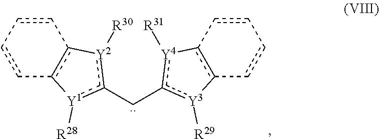

wherein each instance of the dashed line (---) represents an optional covalent bond; wherein each of Y¹⁻⁴ is independently selected from N, NH, C, or CH; wherein each of R²⁸⁻³¹ is independently selected from hydrogen, halide, aryl, heteroaryl, cycloalkyl, heterocycloalkyl, bicyclic cycloalkyl, or bicyclic heterocycloalkyl.

In a further aspect, the salt of the carbene ligand, when present, has a structure represented by Formula (VII-S):

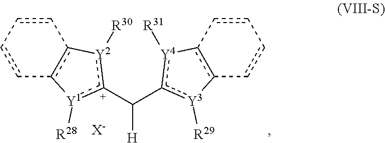

wherein each instance of the dashed line (---) represents an optional covalent bond; wherein each of Y¹⁻⁴ is independently selected from N, NH, C, or CH; wherein each of R²⁸⁻³¹ is independently selected from hydrogen, halide, aryl, heteroaryl, cycloalkyl, heterocycloalkyl, bicyclic cycloalkyl, or bicyclic heterocycloalkyl; and wherein X is halide, BF₄, or PF₆.

In a further aspect, wherein the transition metal-carbene complex, when present, has a structure represented by Formula (VIII-M):

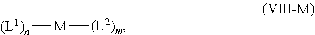

wherein n is an integer ranging from 1-2 and m is an integer ranging from 1-5, provided that when n is 2, m is an integer ranging from 1-4; L¹ is a ligand having a structure represented by the formula:

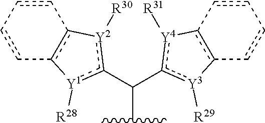

wherein each instance of the dashed line (---) represents an optional covalent bond; wherein each of Y¹⁻⁴ is independently selected from N, NH, C, or CH; wherein each of R²⁸⁻³¹ is independently selected from hydrogen, halide, aryl, heteroaryl, cycloalkyl, heterocycloalkyl, bicyclic cycloalkyl, or bicyclic heterocycloalkyl; wherein M is a Group 8, 9, or 10 transition metal; and wherein each instance of L² is independently —CO or halide.

In one specific aspect, the transition metal-N-heterocyclic carbene complex, when present, has a structure represented by Formula (IX-M):

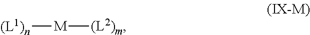

wherein n is an integer ranging from 1-2 and m is an integer ranging from 1-5, provided that when n is 2, m is an integer ranging from 1-4; L¹ is a ligand having a structure represented by the formula:

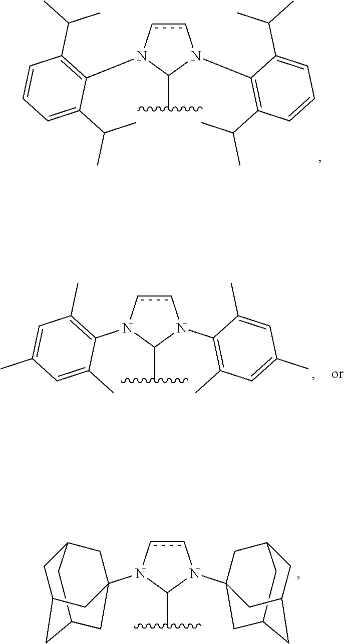

wherein the dashed line (---) represents an optional covalent bond; wherein M is a transition metal selected from nickel, rhodium, or iridium; and wherein each instance of L² is independently —CO or halide. In one specific aspect, M is nickel.

In a further specific aspect, the N-heterocyclic carbene ligand, when present, has a structure represented by Formula (IX), (X), or (XI):

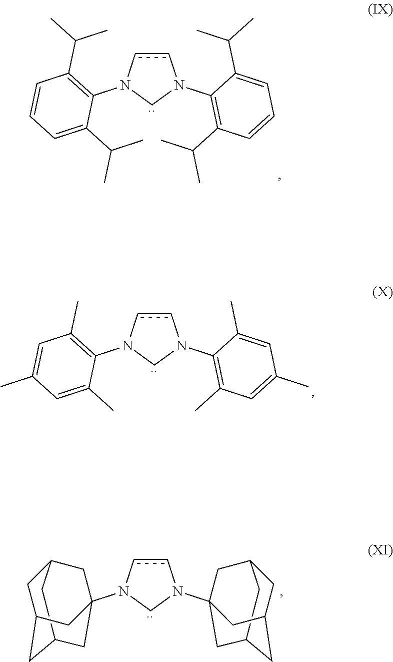

wherein the dashed line (---) represents an optional covalent bond. The N-heterocyclic carbene ligand can be present together with a transition metal compound comprising nickel, rhodium, or iridium. In one aspect, the N-heterocyclic carbene ligand can be present together with a transition metal compound comprising nickel.

In a further specific aspect, the salt of the N-heterocyclic carbene ligand, when present, has a structure represented by Formula (IX-S), (X-S), or (XI-S):

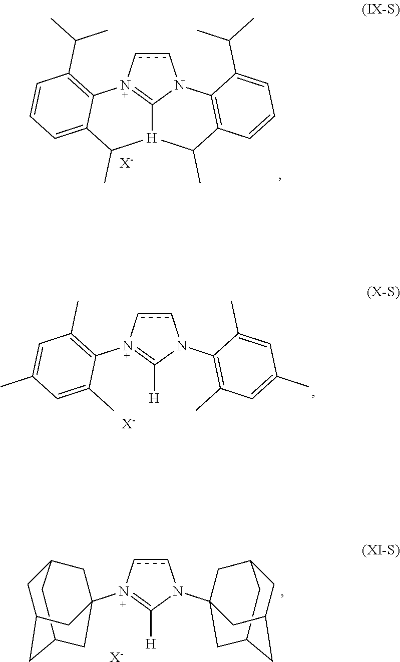

wherein the dashed line (---) represents an optional covalent bond; and wherein X is halide, BF₄, or PF₆. The salt of the carbene ligand can be present with a transition metal compound comprising nickel, rhodium, or iridium. In one aspect, the salt of the carbene ligand can be present with a transition metal compound comprising nickel.

Specific non-limiting examples of transition metal-N-heterocyclic carbene complexes, when present in the catalyst system, include those corresponding to Formula (X-M):

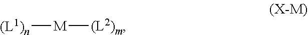

wherein n is an integer ranging from 1-2 and m is an integer ranging from 1-5, provided that when n is 2, m is an integer ranging from 1-4; L¹ is a ligand having a structure represented by the formula:

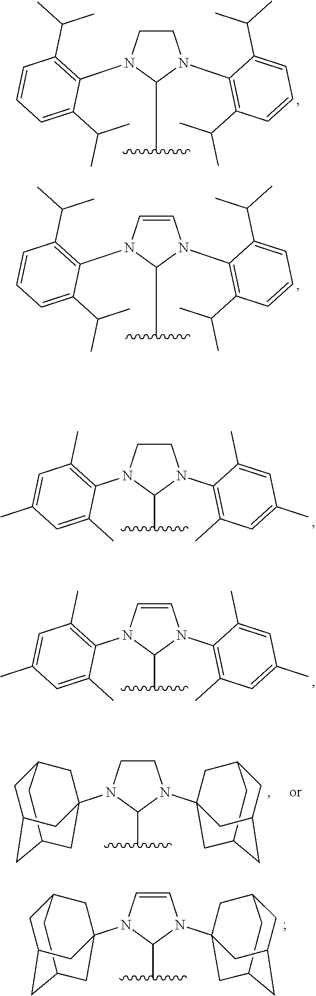

wherein M is a Group 8, 9, or 10 transition metal, such as a transition metal selected from nickel, rhodium, or iridium; and wherein each instance of L² is independently —CO or halide. In one specific aspect of Formula (X-M), n is 1, M is nickel, m is 3, and L² is —CO. In another specific aspect of Formula (X-M), n is 1, M is rhodium, m is 3, two instances of L² are —CO, and one instance of L² is halide, e.g., —I.

More generally, in some aspects of the formulae described herein corresponding to the following structure:

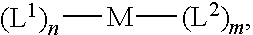

n can be an integer ranging from 1-2, and m can be an integer ranging from 1-3 when M is a Group 10 transition metal such as nickel. In other instances, n can be an integer ranging from 1-2, and m can be an integer ranging from 1-5, provided that when n is 2, m is an integer ranging from 1-4; L¹ can be any of the described ligands; and M can be a Group 8 or 9 transition metal.

Specific non-limiting examples of N-heterocyclic carbenes, when present in the catalyst system, include the following:

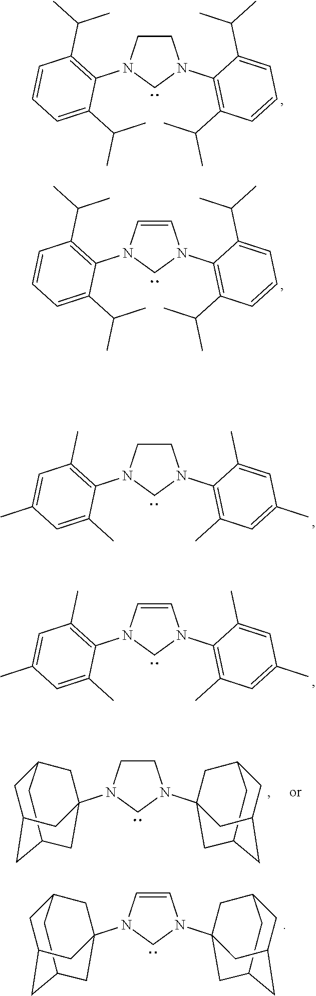

Such carbene ligands, when present, can be present together with a suitable transition metal compound such as NiI₂, NiCl₂ Ni(OAc)₂, IrI₃, IrCl₃, Ir(OAc) 3, RhI₃, RhCl₃, or Rh(OAc)₃.

Specific non-limiting examples of salts of the carbene ligand, when present, include the following:

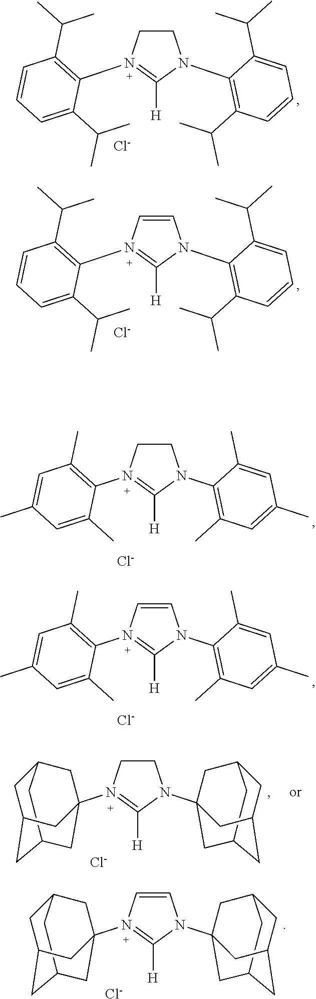

Such salts, when present, can be present together with a suitable transition metal compound such as NiI₂, NiCl₂ Ni(OAc)₂, IrI₃, IrCl₃, Ir(OAc) 3, RhI₃, RhCl₃, or Rh(OAc)₃.

In a further aspect, the salt of the carbene ligand, when present, can be one of the following salts:

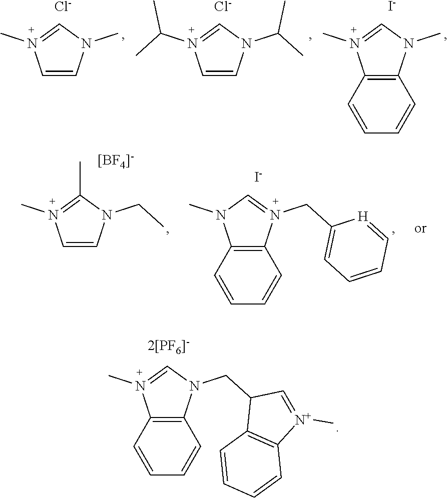

Any of the above salts can be used in their neutral carbene form or as a transition metal-carbene complex, as will be appreciated from the discussion and examples above.

Other specific examples of N-heterocyclic carbenes can be found in Han Vinh Huynh, “Electronic Properties of N-Heterocyclic Carbenes and Their Experimental Determination,” Chem. Rev. 2018, 118, 9457-9492, which is incorporated by reference into this application for its teaching of N-heterocyclic carbenes.

b. Halide Promoters and Other Optional Additives

The catalyst systems in general comprise at least one halide source which can serve as a halide promoter. A suitable example is methyl iodide, Lil, or N-methyl-pyridinium iodide. The alkyl halide serving as the halide promoter can in some aspects be present in the catalyst system prior to carbonylation at a 100:1 molar ratio relative to the carbene catalyst or transition metal compound component of the catalyst precursor that also comprises the carbene ligand or a salt thereof. In other aspects, the alkyl source serving as the halide promoter can be present in the catalyst system prior to carbonylation at a 1:1-200:1 molar ratio relative to the catalyst or transition metal compound component of the catalyst precursor that also comprises the carbene ligand or a salt thereof, in other words, about 1-200 equivalents of the halide source relative to the carbene complex or transition metal compound.

Other additives can in some aspects also be present in the catalyst system. Examples include various halide salts such as lithium iodide or lithium acetate in addition to another halide source. Such additives can be present in the catalyst system prior to carbonylation at a 1:1-200:1 molar ratio relative to the carbene catalyst or transition metal compound component of the catalyst precursor that also comprises the carbene ligand or a salt thereof, in other words, about 1-200 equivalents of additive such as Lil relative to the carbene complex or transition metal compound.

### C. Examples

The following examples are put forth so as to provide those of ordinary skill in the art with a complete disclosure and description of how the methods and products claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. The Examples are provided herein to illustrate the invention, and should not be construed as limiting the invention in any way.

### 1. EXPERIMENTAL DETAILS

General Consideration. All manipulation was carried out using standard Schlenk or glovebox techniques under a N₂ atmosphere, except as noted. CAUTION: When working with CO gas, especially under high pressure, the use of CO monitor is highly recommended. These experiments were conducted with a personal monitor worn on the researcher's lab coat at all times and an additional monitor placed near the regulator of the CO cylinder. CAUTION: Extremely toxic Ni(CO)₄ could be potentially generated in this procedure. Ni(CO)₄ is volatile (b.p. 43° C.), therefore the experiment must be conducted in a well-ventilated fume hood. Any gas and solution that could potentially contained Ni(CO)₄ was quenched with a solution of iodine in acetone. 1,3-bis(2,6-diisopropylphenyl)imidazol-2-ylidene nickel tricarbonyl [Dorta, R.; Stevens, E. D.; Scott, N. M.; Costabile, C.; Cavallo, L.; Hoff, C. D.; Nolan, S. P. Steric and Electronic Properties of N-Heterocyclic Carbenes (NHC): A Detailed Study on Their Interaction with Ni(CO) 4. J. Am. Chem. Soc. 2005, 127 (8), 2485-2495.] and 1,3-bis(1-adamantyl)imidazol-2-ylidene nickel dicarbonyl [Dorta, R.; Stevens, E. D.; Hoff, C. D.; Nolan, S. P. Stable, Three-Coordinate Ni(CO) 2 (NHC) (NHC═N—Heterocyclic Carbene) Complexes Enabling the Determination of Ni—NHC Bond Energies. J. Am. Chem. Soc. 2003, 125 (35), 10490-10491.] were prepared according to literature procedures.

Catalysis using 30 mL stainless steel multireactor system. All catalytic loadings were performed in a glovebox with degassed solvent. A stock solution of the desired catalyst was prepared such that 2 mL were added to the glass liner in each reactor to reach the desired catalyst concentration (typically 5 mM). For example, 240 mg of (IPr)Ni(CO)₃ was dissolved in 12 mL of methyl propionate and 2 mL of this stock solution as added of the six reactors. Next, a stock solution of the appropriate additives was added followed by CH₃I. Additional solvent was added to reach a final volume of 15 mL. For effective heat transfer and prevention of solvent reflux, additional 5 mL solvent was added to the reactor body to fill space between the glass liner and the reactor body. The reactor and vessel heads were sealed in the box under N₂ atmosphere, and the sealed reactors were connected to the multireactor system. The reactor manifold was purged with CO and each vessel was subjected to three pressurization-venting cycles (approximately 10 bar) to ensure full replacement of the N₂ headspace with CO. The vessels were then pressurized as desired and the temperature and stirring (500 rpm) were set utilizing the SpecView 32 software. The t=0 of the reaction was chosen as the time when the reactor vessels reached their set temperature. After the given reaction time, the vessels were allowed to cool to approximately 80° C., then placed further cooled in an ice bath. The pressure was vented through the solution of iodine in acetone to quench any possible Ni(CO) 4. Once vented, hexamethyldisiloxane (HMDSO) internal standard was directly added to the reaction mixture using syringe. An aliquot was removed and placed in an NMR tube with CD₂Cl₂. ¹H NMR spectra were obtained utilizing a delay time of 5 seconds per scan and the concentration of anhydride, MeOAc and AcOH was determined by integration relative to HMDSO internal standard. Turnover number (TON) values were calculated as the moles of product divided by the moles of precatalyst added to the reactor. The NMR analysis revealed formation of scrambled anhydrides and hydrolyzed products; not only acetic-propionic anhydride, but also acetic anhydride and propionic anhydride are observed. The propionic anhydride is not obtained from carbonylation, but through anhydride acyl interchange. Because the total amount of acetyl-CH₃ remains unchanged during anhydride acyl interchange, total amount of anhydride was calculated from the methyl peaks (δ 2.23). The amount of methyl acetate (δ 2.06) and acetic acid (δ 2.10) are also calculated and summed to total turnover number of acetyls, TONₜₒₜ.

Catalysis using a 300 mL C-276 Hastelloy autoclave. In a typical reaction, Ni(OAc)₂·4H₂O (0.75 mmol) and five equivalents of the carbene ligand (IPr or IPr·HCl) were loaded in a vial in a nitrogen-purged box. In a separate bottle was loaded methyl acetate (182 g, 2462 mmol) or methyl isobutyrate (174 g, 1703 mmol) and methyl iodide (10.6 g, 75 mmol). Under air, the solids were transferred to a 300 mL Parr Hastelloy autoclave. The vial was rinsed with the methyl ester/methyl iodide mixture, and the rest of the liquids were then loaded into the reactor. The autoclave was sealed with an overhead stirrer (standard 4 pitch blade impeller) and pressure tested to 90 bar with N₂ gas. The nitrogen was vented, and the reactor was subsequently pressurized with 10 bar CO before venting. After three cycles of CO pressurization/venting, the reactor was once more pressurized to 10 bar CO or 10 bar of a 60:40 mixture of CO:H₂. Stirring was turned on to (500 rpm) and the mixture was heated to 200° C., at which point the pressure was increased to 50 bar CO or 83 bar of a 60:40 mixture of CO:H₂. This was considered time t=0. Pressure was maintained throughout the reaction using a surge tank (for gas uptake measurement) and a Tescom Pressure Controller (a pressure control valve which is controlled via DCS). After 15 h, the reaction was allowed to cool down and subsequently vented through two sequential scrubbers of 8 wt % iodine in isopropanol and 10 wt % caustic, respectively. The reaction was re-pressurized with 10 bar N₂, and vented through the scrubbers once more. The reaction mixture was then transferred to a tared bottle, and the products were quantified by weight % GC. Standards of different components were purchased or independently prepared, and used to determine response factors for GC calibration. Some reactions were monitored in situ using a hollow autoclave equipped with a flexible DST fiber-to-Sentinel conduit, and either a DiComp or SiComp probe. The probe served as the bottom of the hollow autoclave. Data was collected using a ReactIR 45m with a MTC detector and iC IR software.

### 2. CATALYTIC CARBONYLATION OF METHYL PROPIONATE

Catalytic reactions were conducted using steel pressure reactors containing glass liners. Typical reaction conditions consist of 0.075 mmol of the catalyst Ni(NHC)(CO)₃ (NHC ligands shown in Table 1, along with comparison ligand PPh₃), 7.5 mmol methyl iodide, and 20 mL of methyl propionate heated to 200° C. under 50 bar CO for 15 hours. The total yield of carbonylation products (acetyls) was in the range of 7-25 mmol (100-300 turnovers).

TABLE 1
NHC Ligands and Nickel Catalysts

| Abbreviation | Structure |
|---|---|
| IPr | 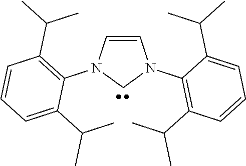 [SMILES: CC(C)c1cccc(C(C)C)c1N1C=CN(c2c(C(C)C)cccc2C(C)C)C1] |
| SIPr | 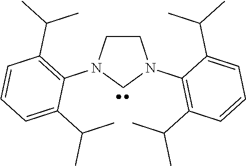 [SMILES: CC(C)c1cccc(C(C)C)c1N1CCN(c2c(C(C)C)cccc2C(C)C)C1] |
| IMes | 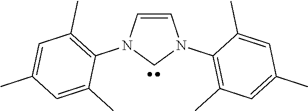 |
| IAd | 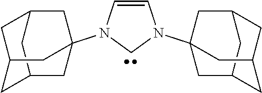 [SMILES: C1=CN(C23CC4CC(CC(C4)C2)C3)CN1C12CC3CC(CC(C3)C1)C2] |
| IPr•HCl | 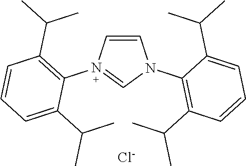 |
| SIPr•HCl | 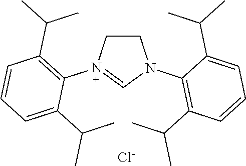 |
| IMes•HCl | 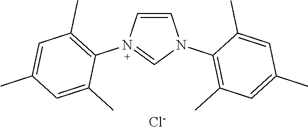 |
| PPh₃ | 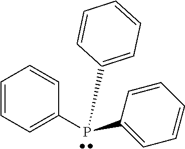 |

The catalytic reaction is shown below in Scheme 1.

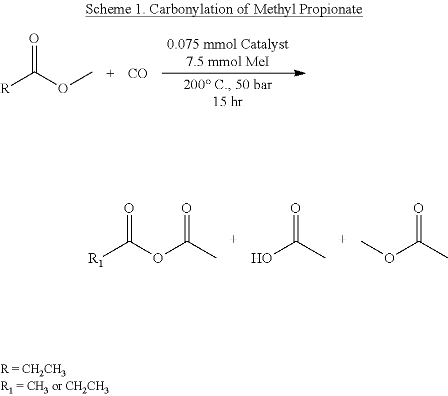

Table 2 shows a summary of typical reaction yields and turnover numbers (TONs). This comparison shows that (1) if desired, the free NHC ligand can be added along with an appropriate nickel precursor, rather than using a synthesized Ni—NHC complex; and (2) the NHC-based catalysts give higher turnover numbers (TONs) than the state-of-the-art triphenylphosphine (PPh₃) based catalysts.

TABLE 2
Reaction Yields and Turnover Number (TON)

| | | | | | Total |
|---|---|---|---|---|---|
| | Anhydride | AcOH | MeOAc | Total | carbonylation |
| | TON | TON | TON | TON | yield (%) |
| NiI₂ + PPh₃ | 31 | 0 | 80 | 111 | 4.0 |
| Ni(PPh₃)₂(CO)₂ | 75 | 10 | 23 | 108 | 3.9 |
| (IPr)Ni(CO)₃ | 179 | 27 | 11 | 218 | 7.9 |
| (IPr)Ni(CO)₃ + | 507 | 41 | 55 | 603 | 21.8 |
| IPr | | | | | |
| Ni(OAc)₂ + IPr | 498 | 16 | 173 | 685 | 24.8 |
| Ni(OAc)₂ + | 565 | 0 | 192 | 755 | 27.3 |
| IPr•HCl | | | | | |
| Ni(IAd)(CO)2 | 100 | 16 | 25 | 141 | 5.1 |
| 0.025 mmol | 980 | 64 | 468 | 1509 | 18.2 |
| Ni(OAc)₂ + | | | | | |
| 0.15 mmol | | | | | |
| IPr; 0.01 | | | | | |
| mol % cat. | | | | | |
| loading | | | | | |

### 3. VARYING REACTION CONDITIONS AND LIGAND EQUIVALENTS FOR CARBONYLATION OF METHYL PROPIONATE

The pressure of CO (P_{CO}) was varied for the catalytic reaction shown in Scheme 2.

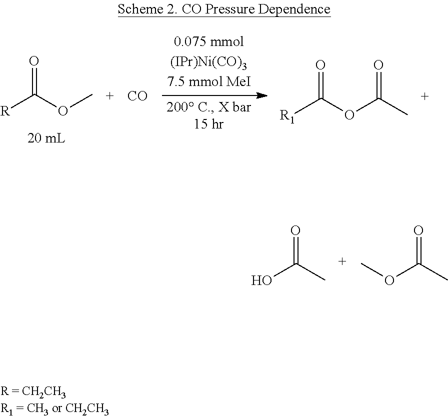

Methyl iodide concentration was varied for the catalytic reaction shown in Scheme 3.

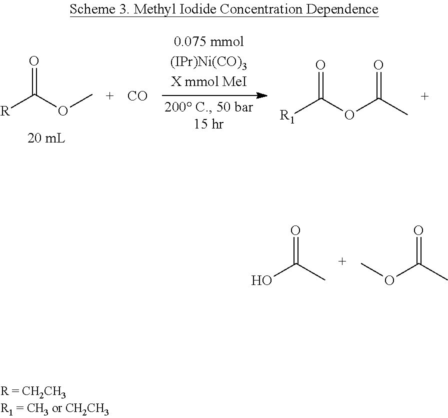

The number of free IPr ligands was varied for the catalytic reaction shown in Scheme 4.

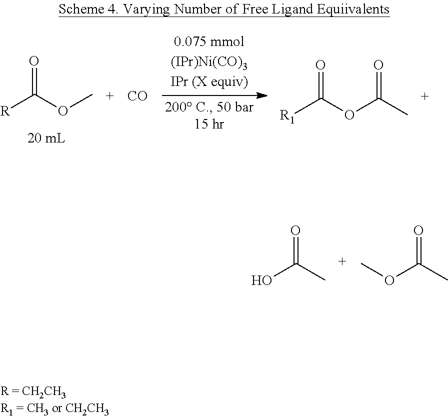

Reaction time was varied for the catalytic reaction shown in Scheme 5.

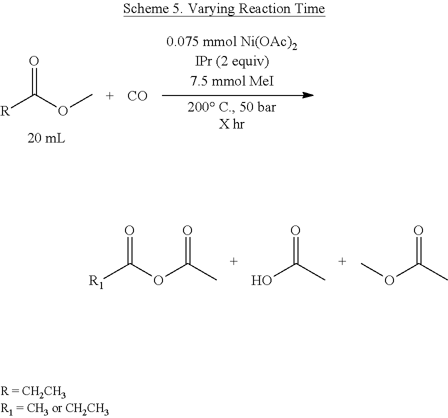

The effect of temperature dependence on catalytic carbonylation was studied using the reaction conditions shown in Scheme 6.

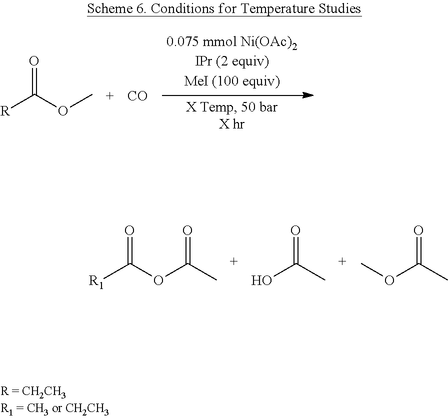

The following tables summarize results obtained by varying carbon monoxide pressure (Table 3), methyl iodide concentration (Table 4), ligand equivalents (Table 5), reaction time (Table 6), and temperature (Table 7). These results show that the reaction rate starts to slow down below 30 atm CO and that increasing the methyl iodide concentration increases the yield of products. Including excess free ligand has a beneficial effect, giving over 400 turnovers and over 15% yield of acetyl products. The experiments varying reaction time show that the catalyst does not rapidly decompose.

TABLE 3
CO Pressure Dependence (See Scheme 2)

| | | | | | Total |
|---|---|---|---|---|---|
| | Anhydride | AcOH | MeOAc | Total | carbonylation |
| P(CO) | TON | TON | TON | TON | yield (%) |
| 20 | 121 | 21 | 4 | 146 | 5.3 |
| 25 | 133 | 23 | 6 | 162 | 5.8 |
| 30 | 192 | 31 | 8 | 231 | 8.3 |
| 35 | 171 | 36 | 6 | 213 | 7.7 |
| 40 | 172 | 35 | 5 | 211 | 7.6 |
| 50 | 188 | 37 | 7 | 232 | 8.4 |

TABLE 4
Methyl Iodide Concentration Dependence (See Scheme 3)

| | | | | | Total |
|---|---|---|---|---|---|
| | Anhydride | AcOH | MeOAc | Total | carbonylation |
| CH₃I/Ni | TON | TON | TON | TON | yield (%) |
| 0 | 1 | 0 | 1 | 2 | 0.1 |
| 1 | 3 | 0 | 3 | 6 | 0.2 |
| 10 | 6 | 0 | 5 | 11 | 0.4 |
| 50 | 39 | 2 | 57 | 98 | 3.5 |
| 100 | 187 | 24 | 5 | 217 | 7.8 |
| 200 | 545 | 58 | 67 | 670 | 24.2 |

TABLE 5
Varying Number of Free Ligand Equivalents (See Scheme 4)

| | | | | | | Total |
|---|---|---|---|---|---|---|
| | | Anhydride | AcOH | MeOAc | Total | carbonylation |
| P(CO) | IPr/Ni | TON | TON | TON | TON | yield (%) |
| 30 | 0 | 192 | 31 | 8 | 231 | 8.3 |
| 30 | 1 | 163 | 25 | 275 | 463 | 16.7 |
| 30 | 5 | 391 | 36 | 49 | 476 | 17.2 |
| 30 | 10 | 129 | 18 | 268 | 414 | 15.0 |
| 50 | 0 | 188 | 37 | 7 | 232 | 8.4 |
| 50 | 1 | 507 | 41 | 55 | 603 | 21.8 |
| 50 | 5 | 485 | 44 | 341 | 870 | 31.4 |
| 50 | 10 | 304 | 32 | 368 | 704 | 25.4 |

TABLE 6
Varying Reaction Time (See Scheme 5)

| | | | | | Total |
|---|---|---|---|---|---|
| Time | Anhydride | AcOH | MeOAc | Total | carbonylation |
| (h) | TON | TON | TON | TON | yield (%) |
| 3 | 82 | 6 | 12 | 100 | 3.6 |
| 7.5 | 95 | 15 | 8 | 116 | 4.2 |
| 15 | 179 | 27 | 11 | 218 | 7.9 |
| 30 | 549 | 45 | 115 | 707 | 25.5 |

TABLE 7
Results from Temperature Studies (See Scheme 6)

| | | | | | Total |
|---|---|---|---|---|---|
| T | Anhydride | AcOH | MeOAc | Total | carbonylation |
| (° C.) | TON | TON | TON | TON | yield (%) |
| 50 | 7 | 0 | 0 | 7 | 0.3 |
| 80 | 6 | 0 | 0 | 6 | 0.2 |
| 110 | 8 | 0 | 0 | 8 | 0.3 |
| 140 | 10 | 0 | 1 | 11 | 0.4 |
| 160 | 33 | 11 | 0 | 44 | 1.6 |
| 180 | 88 | 17 | 0 | 105 | 3.8 |
| 200 | 179 | 27 | 11 | 218 | 7.9 |
| 220 | 227 | 49 | 44 | 320 | 11.6 |

### 4. NHC LIGAND STUDIES

Studies examining the structure of N-heterocyclic carbene ligands were conducted for the catalytic reaction shown in Scheme 7.

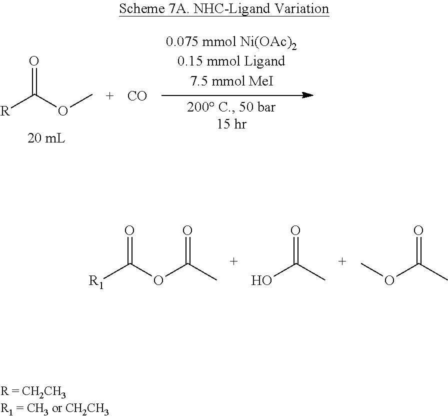

Scheme 7B compares five NHC ligands, both as the free base imidazolylidene (NHC) and in the protonated imidazolium form. Imidazolium and imidazolylidene compounds can be used interchangeably. This is advantageous because imidazolium salts, such as IPr·HCl, are air stable and easier to prepare and handle than the imidazolylidene compounds. Bidentate ligands were also examined.

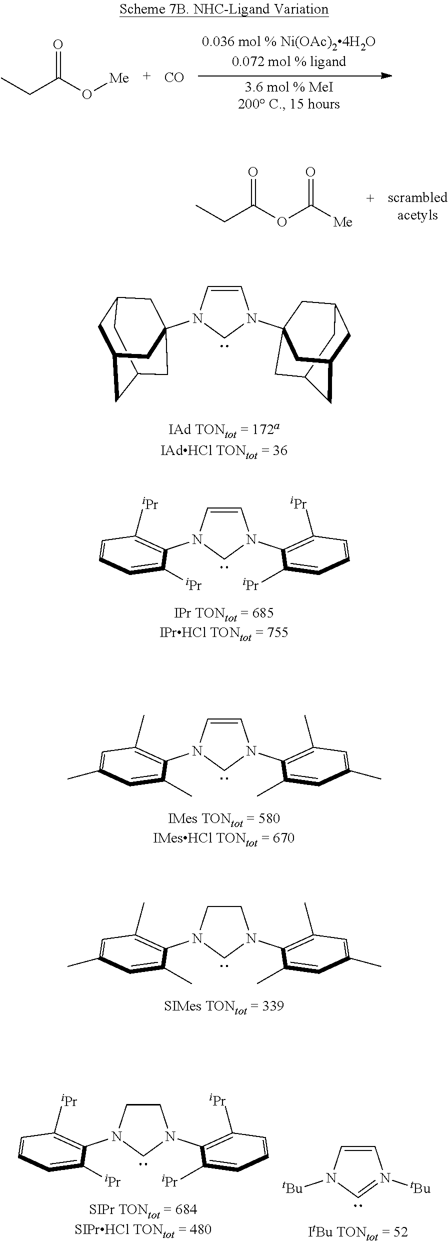

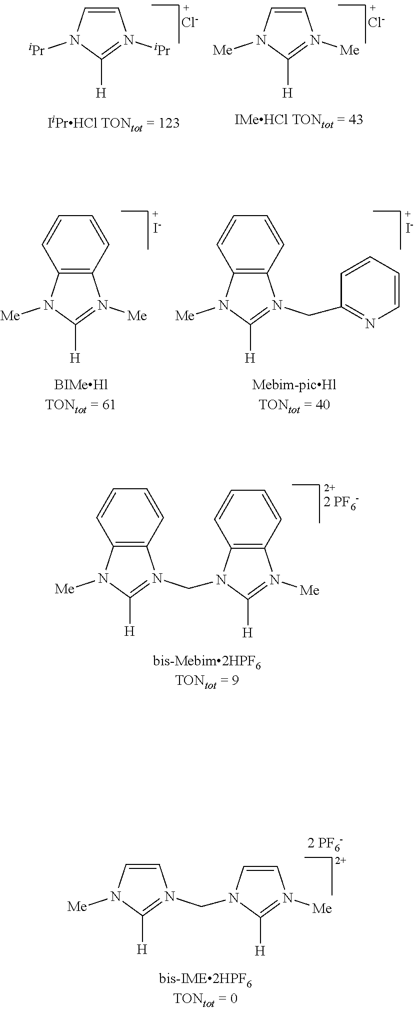

### 5. HALIDE PROMOTER STUDIES

Various halide promoters were examined for the standard catalytic reaction conditions shown in Scheme 8.

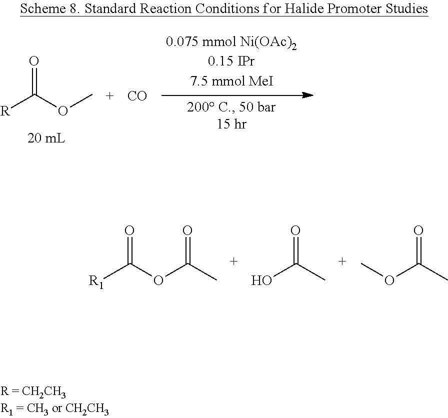

Table 8 shows that using NiI₂ in place of Ni(OAc)₂ leads to an increase in activity. Even higher activity is observed when 0.75 mmol Lil is added to the reaction mixture.

TABLE 8
Deviation from Standard Reaction Conditions

| | Anhydride | AcOH | MeOAc | Total | Total carbonylation |
|---|---|---|---|---|---|
| | TON | TON | TON | TON | yield (%) |
| No | 498 | 16 | 173 | 685 | 24.8 |
| additive | | | | | |
| NiI₂ | 646 | 0 | 74 | 720 | 26.0 |
| LiI (10 | 621 | 0 | 157 | 776 | 28.0 |
| equiv) | | | | | |
| LiI (100 | 306 | 44 | 454 | 801 | 28.9 |
| equiv) | | | | | |
| NiI₂ + | 330 | 52 | 504 | 883 | 31.9 |
| LiI (100 | | | | | |
| equiv) | | | | | |
| No CH₃I | 1 | 0 | 1 | 2 | 0.07 |

### 6. LARGE-SCALE CATALYTIC CARBONYLATION

Catalytic methyl ester carbonylation was also successfully conducted in larger, 300 mL autoclaves equipped with a surge tank to maintain a constant pressure of CO throughout the reaction, according to Scheme 9.

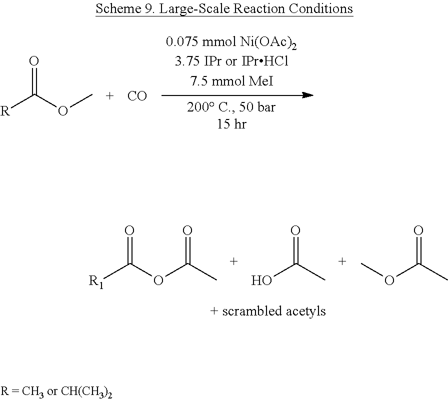

In some instances, the reactor was equipped with an infrared spectroscopy probe for in situ monitoring of the reaction mixture. Typical reaction conditions consisted of 0.75 mmol of nickel (II) acetate precursor, 3.75 mmol of IPr ligand, 75 mmol of methyl iodide, and either 182 g of methyl acetate or 174 g of methyl isobutyrate, heated to 200° C. under 50 bar CO for 15 hours. These reactions afforded a range of 417-631 turnovers.

Using the nickel catalyst, in the presence of H₂, the reaction afforded comparable yields and no unwanted hydrogenation products, highlighting the flexibility of using less pure CO sources. Using the protonated chloride salt of IPr yielded slightly lower turnovers compared to the free ligand, but still exhibited appreciable catalytic activity. In this run, the (IPr)Ni(CO) 3 intermediate was spectroscopically observed at the start of the reaction, indicating that the protonated chloride salt of IPr underwent deprotonation and the free ligand was able to bind to the metal center. In all experiments with methyl isobutyrate, symmetric isobutyric anhydride was also detected and quantified, ranging from 34 to 220 mmol, indicative of anhydride acyl interchange taking place over the course of the reaction.

Results are shown in Table 9.

TABLE 9
Methyl Isobutyrate Carbonylation in Larger Reactors

| | Run (Deviation | | | | | |
|---|---|---|---|---|---|---|
| | from original | MeOAc | AcOH | Anhydrides | | Yield |
| Ester | conditions) | TON | TON | TON | TONₜₒₜ | % |
| Methyl | 1 (none) | — | — | 631 | 631^{a} | 19.2 |
| acetate | | | | | | |
| Methyl | 2 (none) | — | — | 463 | 463^{a} | 14.1 |
| acetate | | | | | | |
| Methyl | 3 (none) | 340 | 89 | 194 | 623^{b} | 27.4 |
| isobutyrate | | | | | | |
| Methyl | 4 (none) | 274 | 62 | 212 | 548^{b} | 24.1 |
| isobutyrate | | | | | | |
| Methyl | 5 (with 60:40 | 269 | 222 | 135 | 627^{b} | 27.6 |
| isobutyrate | CO:H₂)^{c} | | | | | |
| Methyl | 6 (3.75 mmol | 164 | 106 | 146 | 417^{b} | 18.3 |
| isobutyrate | IPr•HCl in place of | | | | | |
| | free IPr) | | | | | |

^{a}Calculated as the mmol of acetic anhydride exclusively divided by mmol of catalyst (0.75), as AcOH could derive from MeOAc hydrolysis and not carbonylation.
^{b}Calculated as the sum of mmol of acetyl-containing species (MeOAc, AcOH, acetic anhydride, and mixed acetic isobutyric anhydride) divided by mmol of catalyst (0.75).
^{c}CO partial pressure of 50 bar.

### 7. METAL SCREENING

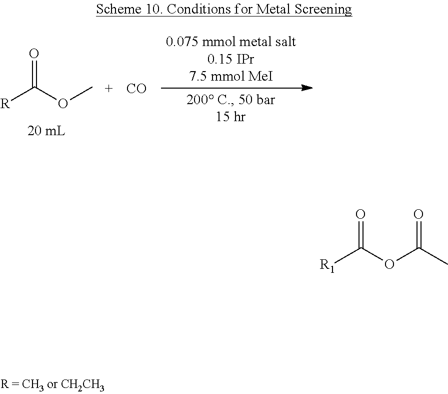

TABLE 10
Results from metal screening

| | | | Total |
|---|---|---|---|
| | | Anhydride | carbonylation |
| Ester | Metal precursor | TON | yield (%) |
| Methyl | — | 0 | 0 |
| propionate | | | |
| Methyl acetate | — | 0 | 0 |
| Methyl acetate | Pd(OAc)₂ | 152 | 4.5 |
| Methyl acetate | Rh(acac)(CO)₂ | 509 | 15.2 |
| Methyl acetate | RuCl₃•3H2O | 6 | 0.2 |
| Methyl acetate | PtCl₂(CO)₂ | 21 | 0.6 |
| Methyl acetate | Co(OAc)₂•4H₂O | 7 | 0.2 |
| Methyl acetate | Fe(OAc)₂ | 37 | 1.1 |
| Methyl acetate | IrCl₃•3H₂O | 8 | 0.3 |

Other group 8-10 metals were screened and the data is shown in Table 10. Without metals, there were no detectable carbonylation products. Using a rhodium catalyst instead of nickel under identical conditions afforded similar TON.

### 8. REFERENCES

(1) Gauthier-Lafaye, J.; Perron, R. U.S. Pat. No. 4,353,844, 1982.
(2) Rizkalla, N.; Vale, R.; Wells, H. N. U.S. Pat. No. 4,356,320, 1982.
(3) Isshiki, T.; Kijima, Y.; Kondo, T. U.S. Pat. No. 4,544,511, 1985.
(4) Rizkalla, N. Acetic Acid Production via Low-Pressure, Nickel-Catalyzed Methanol Carbonylation. In ACS Symposium Series; 1987; pp 61-76.
(5) Erpenbach, H.; Gradl, R.; Jagers, E.; Seidel, A. Australian Patent, 641056, 1990.
(6) Kelkar, A. A.; Ubale, R. S.; Chaudhari, R. V. Carbonylation of Methyl Acetate to Acetic Anhydride Using Homogeneous Nickel Complex Catalyst. J. Mol. Catal. 1993, 80 (1), 21-29.
(7) Ubale, R. S.; Kelkar, A. A.; Chaudhari, R. V. Carbonylation of Ethanol Using Ni-Isoquinoline Complex Catalyst: Activity and Selectivity Studies. J. Mol. Catal. A Chem. 1997, 118 (1), 9-19.
(8) Gong, J.; Fan, Q.; Jiang, D. Nickel-Catalyzed Carbonylation of Methyl Acetate to Acetic Anhydride. J. Mol. Catal. A Chem. 1999, 147 (1-2), 113-124.
(9) Moser, W. R.; Marshik-Guerts, B. J.; Okrasinski, S. J. The Mechanism of the Phosphine-Modified Nickel-Catalyzed Acetic Acid Process. J. Mol. Catal. A Chem. 1999, 143 (1-3), 71-83.
(10) Kanel, J.; Okrasinski, S. U.S. Pat. No. 5,900,504, 1999.
(11) Moser, W. R.; Marshik-Guerts, B. J.; Okrasinski, S. J. An in Situ CIR-FTIR Investigation of Process Effects in the Nickel Catalyzed Carbonylation of Methanol. J. Mol. Catal. A Chem. 1999, 143 (1-3), 57-69.
(12) Sarkar, B. R.; Chaudhari, R. V. Carbonylation of Alkynes, Alkenes and Alcohols Using Metal Complex Catalysts. Catal. Surv. from Asia 2005, 9 (3), 193-205.
(13) Sabater, S.; Menche, M.; Ghosh, T.; Krieg, S.; Rück, K. S. L.; Paciello, R.; Schäfer, A.; Comba, P.; Hashmi, A. S. K.; Schaub, T. Mechanistic Investigation of the Nickel-Catalyzed Carbonylation of Alcohols. Organometallics 2020, 39, 870-880.

It will be apparent to those skilled in the art that various modifications and variations can be made without departing from the scope or spirit of this disclosure. Other embodiments will be apparent to those skilled in the art from consideration of the specification and practice disclosed herein. It is intended that the specification and examples be considered as exemplary only, with a true scope and spirit being indicated by the following claims.

1. A method comprising carbonylating an ester in a reactor comprising carbon monoxide or a source thereof in the presence of a catalyst system;
wherein the ester has a structure represented by Formula (I): 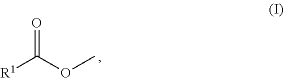
wherein R¹ is hydrocarbyl;
wherein the catalyst system comprises:
a) at least one of:
i) a transition metal-carbene complex; or
ii) a carbene ligand, or a salt thereof, and a transition metal compound; and
b) an halide source.

2. The method of claim 1, wherein the carbon monoxide is present in a syngas composition comprising hydrogen gas.

3. The method of claim 1, wherein the carbon monoxide is present in the reactor at a partial pressure of at least 20 bar.

4. The method of claim 1, wherein the carbon monoxide is present in the reactor at a partial pressure of 20-50 bar.

5. The method of claim 1, wherein R¹ is C1-C20 alkyl.

6. The method of claim 1, wherein R¹ is C1-C10 alkyl.

7. The method of claim 1, wherein R¹ is C2-C3 alkyl.

8. The method of claim 1, wherein the ester is methyl acetate, methyl propionate, methyl butyrate, or methyl isobutyrate.

9. The method of claim 1, wherein carbonylation is carried out at a temperature of at least 50° C.

10. The method of claim 1, wherein carbonylation is carried out at a temperature of at least 180° C.

11. The method of claim 1, wherein carbonylation is carried out at a temperature of at least 200° C.

12. The method of claim 1, wherein the transition metal-carbene complex, when present, is a transition metal-N-heterocyclic carbene complex; or wherein the carbene ligand, when present, is an N-heterocyclic carbene; or wherein the salt of the carbene ligand, when present, is an N-heterocyclic carbene salt.

13. The method of claim 12, wherein the transition metal-N-heterocyclic carbene complex, when present, or the transition metal compound, when present, comprises a Group 8, 9, or 10 transition metal.

14. The method of claim 12, wherein the transition metal-N-heterocyclic carbene complex, when present, or the transition metal compound, when present, comprises a transition metal selected from nickel, rhodium, or iridium.

15. The method of claim 1, wherein transition metal-carbene complex, when present, or the transition metal compound, when present, comprises a Group 8, 9, or 10 transition metal.

16. The method of claim 1, wherein transition metal-carbene complex, when present, or the transition metal compound, when present, comprises nickel, rhodium, or iridium.

17. The method of claim 1, wherein, prior to carbonylation, the ester and the transition metal-carbene complex or the transition metal compound are present in the reactor at a molar ratio ranging from 100:1 to 10,000:1 (ester:transition metal-carbene complex or transition metal compound).

18. The method of claim 1, wherein, prior to carbonylation the carbene ligand or the salt thereof and the transition metal-carbene complex or the transition metal compound are present in the reactor at a molar ratio ranging from 1:1 to 10:1 (carbene ligand or salt thereof:transition metal-carbene complex or transition metal compound).

19. The method of claim 1, wherein the carbene ligand, when present, has a structure represented by Formula (II): 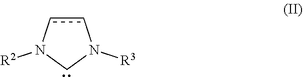
wherein the dashed line (---) represents an optional covalent bond;
and wherein R² and R³ are independently selected from aryl, heteroaryl, cycloalkyl, heterocycloalkyl, bicyclic cycloalkyl, or bicyclic heterocycloalkyl; or
wherein the salt of the carbene ligand, when present, has a structure represented by Formula (II-S): 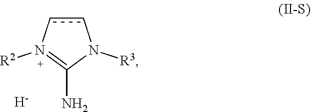
wherein the dashed line (---) represents an optional covalent bond;
and wherein R² and R³ are independently selected from aryl, heteroaryl, cycloalkyl, heterocycloalkyl, bicyclic cycloalkyl, or bicyclic heterocycloalkyl; and
wherein X is halide, BF₄, or PF₆; or
wherein the transition metal-carbene complex, when present, has a structure represented by Formula (II-M): 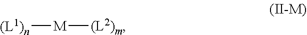
wherein n is an integer ranging from 1-2 and m is an integer ranging from 1-5, provided that when n is 2, m is an integer ranging from 1-4;
L¹ is a ligand having a structure represented by the formula: 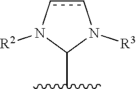
wherein the dashed line (---) represents an optional covalent bond;
wherein R² and R³ are independently selected from aryl, heteroaryl, cycloalkyl, heterocycloalkyl, bicyclic cycloalkyl, or bicyclic heterocycloalkyl;
wherein M is a Group 8, 9, or 10 transition metal; and
wherein each instance of L² is independently —CO or halide.

20. The method of claim 1, wherein the carbene ligand, when present, has a structure represented by Formula (III): 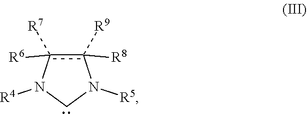
wherein each instance of the dashed line (---) represents an optional covalent bond;
wherein R⁴ and R⁵ are independently selected from aryl, heteroaryl, cycloalkyl, heterocycloalkyl, bicyclic cycloalkyl, or bicyclic heterocycloalkyl; wherein R⁶ and R⁸ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R⁶ and R⁸ can together form an aryl, heteroaryl, cycloalkyl, heterocycloalkyl, bicyclic cycloalkyl, or bicyclic heterocycloalkyl ring; and wherein R⁷ and R⁹, when present, are independently selected from hydrogen, halide, C1-C4 alkyl, aryl, heteroaryl, cycloalkyl, heterocycloalkyl, bicyclic cycloalkyl, or bicyclic heterocycloalkyl; or
wherein the salt of the carbene ligand, when present, has a structure represented by Formula (III-S): 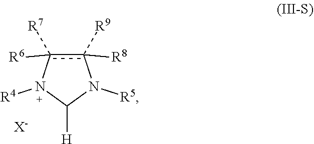
wherein each instance of the dashed line (---) represents an optional covalent bond;
wherein R⁴ and R⁵ are independently selected from aryl, heteroaryl, cycloalkyl, heterocycloalkyl, bicyclic cycloalkyl, or bicyclic heterocycloalkyl; wherein R⁶ and R⁸ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R⁶ and R⁸ can together form an aryl, heteroaryl, cycloalkyl, heterocycloalkyl, bicyclic cycloalkyl, or bicyclic heterocycloalkyl ring; wherein R⁷ and R⁹, when present, are independently selected from hydrogen, halide, C1-C4 alkyl, aryl, heteroaryl, cycloalkyl, heterocycloalkyl, bicyclic cycloalkyl, or bicyclic heterocycloalkyl; and wherein X is halide, BF₄, or PF₆; or
wherein the transition metal-carbene complex, when present, has a structure represented by Formula (III-M): 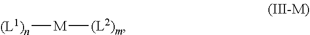
wherein n is an integer ranging from 1-2 and m is an integer ranging from 1-5, provided that when n is 2, m is an integer ranging from 1-4;
L¹ is a ligand having a structure represented by the formula: 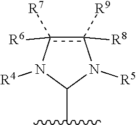
wherein each instance of the dashed line (---) represents an optional covalent bond;
wherein R⁴ and R⁵ are independently selected from aryl, heteroaryl, cycloalkyl, heterocycloalkyl, bicyclic cycloalkyl, or bicyclic heterocycloalkyl; wherein R⁶ and R⁸ are independently selected from hydrogen, halide, C1-C4 alkyl, aryl, heteroaryl, cycloalkyl, heterocycloalkyl, bicyclic cycloalkyl, or bicyclic heterocycloalkyl, or wherein R⁶ and R⁸ can together form an aryl, heteroaryl, cycloalkyl, heterocycloalkyl, bicyclic cycloalkyl, or bicyclic heterocycloalkyl ring; wherein R⁷ and R⁹, when present, are independently selected from hydrogen, halide, C1-C4 alkyl, aryl, heteroaryl, cycloalkyl, heterocycloalkyl, bicyclic cycloalkyl, or bicyclic heterocycloalkyl;
wherein M is a Group 8, 9, or 10 transition metal; and
wherein each instance of L² is independently —CO or halide.

21-64. (canceled)
